(19) United States
(12) Patent Application Publication
(10) Pub. No.: US 2002/0037864 A1
(43) Pub. Date: Mar. 28, 2002

(54) 2'-Propionate clarithromycin dodecyl sulfate and its preparation and pharmaceutical composition containing the same

(76) Inventor: Xinhua Wang, Zhejiang (CN)

Correspondence Address: Madeline I. Johnston, Morrison & Foerster LLP, 755 Page Mill Road, Palo Alto, CA, 94304-1018, US

(21) Appl. No.: 09/886,390

(22) Filed: Jun. 20, 2001

(30) Foreign Application Priority Data
Jun. 20, 2000 (CN) 00117791.5

(51) Int. Cl. A61K 31/7048; C07H 17/08

(52) U.S. Cl. 514/28; 536/7.1

(57) ABSTRACT

The present invention relates to a novel macrolide derivative 2′-propionate-clarithromycin dodecyl sulfate (I), and to a method for preparing the same as well as to a pharmaceutical composition containing the same as antibiotics.

[chemical structure]

### FIELD OF THE INVENTION

The present invention relates to a novel macrolide derivative 2′-propionate -clarithromycin dodecyl sulfate (I), and to a method for preparing the same as well as to a pharmaceutical composition containing the same as antibiotics.

### BACKGROUND OF THE INVENTION

It is already known that etothromycin, 2′-propionate erythromycin dodecyl sulfate with molecular formula C₄₁H₇₁NO₁₄·C₁₂H₂₆O₄S (CA register number 3521-62-8), is called tasteless erythromycin because it is non-bitterness. As to 2′-propionyl erythromycin (named compound III hereafter), its molecular formula is C₄₁ H₇₁NO₁₄ (CA register number 134-36-1) with structural formula III as follows:

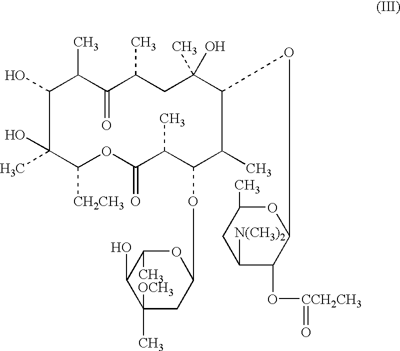

Although compound (III) is less bitterness than erythromycin, it has never been used as medicament but an intermediate of 2′-propionyl erythromycin dodecyl sulfate because its pharmaceutical efficacy is poor relative to that of erythromycin in the form of salt. In fact, compound (III) has never been used as a therapeutic agent up to date, only 2′-propionate erythromycin dodecyl sulfate has been recorded in the Chinese Pharmacopoeia, the American Pharmacopoeia and the European Pharmacopoeia.

Clarithromycin derived by methylating the 6-hydroxyl of erythromycin, has improved pharmaceutical efficacy than that of erythromycin, while its medicinal mechanism is not changed. It is also been deemed that the hydroxyl group in 6′ position is effectively protected by methylation to give a methoxyl group which has reduced activity and stable, so the 2′ hydroxyl group of clarithromycin can be fully propionylized by propionic anhydride with much less side product.

It reported that clarithromycin had already been further modified. For example, Japanese unexamined patent SHO61-200998 disclosed a synthesis method of 2′-propionate clarithromycin (C₄₁H₇₃ NO₁₄ ) [CA register number 107783-86-8](named compound II hereafter).

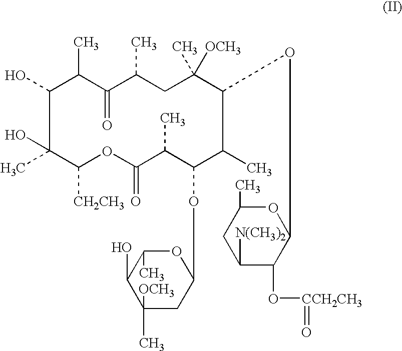

The method for preparing compound (II) described in said application is as follows: clarithromycin is added into solution of anhydrous sodium carbonate in dichloromethane or acetone, then propionic anhydride or propionyl chloride is added thereinto, and resulting product is separated using saturated aqueous solution of sodium carbonate and further extracted by dichloromethane or acetone, the organic phase is then washed extensively with saturated aqueous solution of sodium chloride and then dried with anhydrous magnesium sulfate. The solvent was evaporated to obtain compound (II) 2′-propionate clarithromycin.

However, no such suggestion or teaching is included in said patent application that resultant compound (II) 2′-propionate clarithromycin can be further salified with a salt of dodecyl sulfate and used as pharmaceutical component thereby.

Surprisingly, the inventor of present application find that a novel compound (I) of present application can be prepared by propionyling the 2′-hydroxyl of clarithromycin followed by salified with sodium dodecyl sulfate, which result in that the inherent bitterness of clarithromycin has been eliminated while excellent pharmaceutical efficacy remained.

### SUMMARY OF THE INVENTION

In one aspect of the present invention, a macrolide derivative 2′- propionate clarithromycin dodecyl sulfate is provided, wherein the compound has molecular formula C₄₁H₇₃NO₁₄·C₁₂H₂₆O₄S with structural formula I as follows:

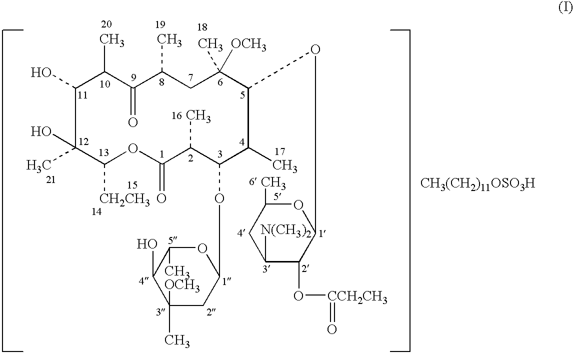

In another aspect of this invention, a method for preparing the above-described compound (I) is provided, which is characterized by propionylizing the 2′-hydroxyl group of clarithromycin to produce 2′-propionyl clarithromycin (lI), followed by reacting it with sodium dodecyl sulfate to form compound (I).

In a third aspect of the present invention, a pharmaceutical composition comprising 2′-propionate clarithromycin dodecyl sulfate used as antibacterials and/or antipyrotics is also provided.

### DETAILED DESCRIPTION OF THE INVENTION

It is contemplated in the present invention the macrolide derivatives, i.e., an acid salt of 2′- propionate clarithromycin, wherein claimed compound has general molecular formula C₄₁H₇₃NO₁₄·R, with structural formula as follows. wherein R is selected from the group consisting of dodecyl sulfate, thiocyanic acid, glucoheptose, ascorbic acid, and fumaric acid. Preferably, R is dodecyl sulfate.

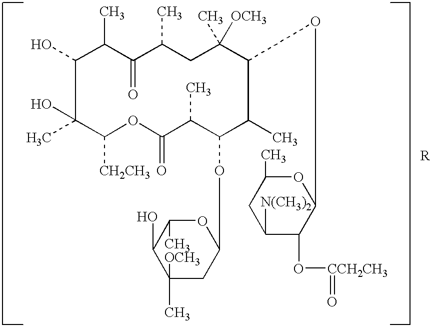

Thus, a macrolide derivative 2′- propionate clarithromycin dodecyl sulfate is provided, wherein compound I has molecular formula C₄₁H₇₃NO₁₄·C₁₂H₂₆O₄S with structural formula I as above-described. In this way, the inherent bitterness of clarithromycin is eliminated from compound (I) without reducing pharmaceutical efficacy. Therefore, compound I of the present invention with comparable efficacy to that of clarithromycin can be developed into an novel antibiotics.

In another aspect of the present invention, a method for preparing the above-described compound (I) is disclosed, which is characterized by propionylizing the 2′-hydroxyl group of clarithromycin to make 2′-propionyl clarithromycin (II) followed by reacting with sodium dodecyl sulfate to form compound (I). Specifically, method of the present invention comprises following steps:

1) adding clarithromycin into a water-soluable non-polar solvent under anhydrous condition, then adding anhydrous propionic anhydride or propionyl chloride thereinto with temperature held at 15 to 45° C., stirring for 0.5 to 3 hours to make clarithromycin fully dissolved and thereby obtaining clear solution of 2′-propionyl clarithromycin,

2) reducing the temperature of solution to about 10-35° C., keeping at the temperature and adding dropwise aqueous solution of sodium dodecyl sulfate into said solution with stirring over 30 to 90 minute, stopping reaction, then

3) filtering with suction to obtain white crystal,

4) concentrating the resulting filtrate and reclaiming solvent, adding 0.2 to 3 volumes distilled water into the concentrated solution followed by refrigerating at 0 to 10° C. over 1-24 hours before filtering said solution with suction to obtain white crystal,

5) combining resultant white crystal in step 3) and 4), then wasing with distilled water extensively, and drying under vacuum condition to obtain compound (I).

In one preferred embodiment of present invention, the method of the present invention for preparaing compound (I) is carried out in “one-pot” method comprising that clarithromycin is esterified with anhydrous propionic anhydride, then it is salified with sodium dodecyl sulfate to yield compound (I). The term “one-pot” method as used herein means a method in which the entire procedure takes place in one reaction container and individual reaction product(s) and/or intermediate(s) at each reaction stage are not necessary to be separated and/or purified.

Because the reaction was performed according to the “one-pot” method, the intermediates need not be separated and purified. When reaction completing, the product is simply refined with water. Consequently, extraction by organic solvent and step for separating organic solvent are eliminated which reduce the risk of environmental pollution and increase the yield of the method of the present invention.

The suitable water-soluble non-polar organic solvent in the method of present invention for preparing compound (I) include, but not limited to, acetone, butanone, cyclohexanone, tetrahydrofuran and the like, and acetone is preferred. When aqueous solution of sodium dodecyl sulfate, which less soluble at room temperature, is prepared, it should be kept in bath of warm water to facilitate sodium dodecyl sulfate dissolve and make resulting solution clear. Optionally, 1 to 3 moles of propionic acid, or sulfuric acid and the like can be added into said aqueous solution of sodium dodecyl sulfate.

When adding said aqueous solution of sodium dodecyl sulfate according to the present method, the rate of dropping should not be too fast, or the color of resulting product will be deepened. However, it should not be too slow which will result in over-esterification. On another hand, the temperature should not be too high, or it will also result in over-esterification and the solvent will run-off.

Consequently, two ways of dropping aqueous solution of sodium dodecyl sulfate, i.e. continuous dropping and intermittent dropping, are suitable and therefore adopted in the present method. The term “continuous dropping” as used herein refers to that dropping is carried out continuously once starting until the reaction solution is entirely added thereinto. The term “intermittent dropping” as used herein refers to that dropping is carried out in non-continuous manner in which dropping will be paused for a certain time period after a portion of reaction solution is added, then restart dropping and the whole reaction solution is added thereinto. There will be one or more time interval as above-described been incorporated into the intermittent dropping procedure of the present invention before all solution is added.

In general, the amount of each reaction agent used in the method of present invention for preparing compound (I) is based on 1 mole clarithromycin as original substrate, correspondingly, anhydrous propionic anhydride or propionyl chloride added is from 0.5 to 2 moles, sodium dodecyl sulfate is from 1 to 4 moles.

Typically, a large quantity of clear crystal will appeared 15 min after aqueous solution of sodium dodecyl sulfate is added dropwise. However, said crystal will disappeared slowly with continuous stirring and dropping. The mixture is stirred continuously for 1 to 3 hours before reaction is completed. Then the mixture is filtered with suction to obtain white crystal. The filtrate is further concentrated and acetone is recovered. 0.2 to 3 volume of distilled water is added into such concentrated solution so as to reduce the solubility of resulting product in the organic solvent in filtrate. The mixture is refrigerated at 0 to 10° C. for 1 to 24 hours till the white needle-shaped crystals is visible on the wall of container. The mixture is again filtered with suction to obtain white crystals. In this way, environmental pollution is significantly reduced and the yield is increased.

The resulting white crystals are pooled and washed 3 to 8 times with distilled water to eliminate the residual impurities in crystals, such as sodium dodecyl sulfate and the like, then it is dried under vacuum condition to obtain white powdery needle-like crystals, i.e. compound (I). The highest yield of the method of present invention is greater than 90%. The purity of such crystals is greater than 95% as high performance liquid chromatography (HPLC) determined.

In a third aspect of the present invention, a pharmaceutical composition comprising 2′-propionate clarithromycin dodecyl sulfate used as antibacterials and/or antipyrotics is also provided.

It is also contemplated that conventional pharmaceutical acceptable excipient and/or carrier can be included into pharmaceutical composition containing 2′-propionate clarithromycin dodecyl sulfate of the present invention. The suitable excipient and/or carrier is well known to skilled person in the art. Depending on specific administration route, the pharmaceutical composition of present invention can be suitably formulated into dry syrup, suspension, tablet and the like. In particular, such pharmaceutical composition of present invention as described can be orally administrated, which, among others, especially suitable to infant and/or child patient. Furthermore, it can be administrated along with food simultaneously.

### BRIEF DESCRIPTION OF THE DRAWINGS

FIG. 1 represents the result of high performance liquid chromatogram of compound (I);

FIG. 2 represents the result of high performance liquid chromatogram of clarithromycin;

FIG. 3 represents the result of infrared spectrogram of compound (I).

FIG. 4 represents theresult of nuclear magnetic resonance (NMR) of compound (I).

To make further illustrate about the compound (I) of present invention and the method for preparing the same, following examples were given which in no ways intend to limit the scope of present invent.

### EXAMPLE 1

Clarithromycin 7.4 g (0.01 mole) is added into flask containing 29 g (0.5 mol) of acetone, mixing, then 2 g (0.015 mole) of anhydrous propionic anhydride is added thereinto with stirring at rate of 120 rpm. The reaction temperature is about 25+ C. Clarithromycin is substantially dissolved over 1 hour. Solution becomes clear 1.5 hour later and the reaction was stopped.

The water bath is moved away, 1.8% (by weight) aqueous solution of sodium dodecyl sulfate 180 ml are added dropwise at room temperature. The dropping was carried out continuously at rate of 3 to 5 ml/ min with rapid stirring (>120 rpm). The reaction remains 2 hours after dropping completed and large quantity of white crystal appears. Recovering white crystal by the following steps: filtration with suction, washing extensively by distilled water and further suction 3 times before it is dried under vacuum condition ( setting temperate <80° C.). In this way, 9.36 g white crystal product is obtained.

The filtrate is further concentrated under reduced pressure to extract organic solvent acetone. After that, 36 ml (0.2 volume) of distilled water is added into concentrated solution, then mixed, and refrigerated at 0-10° C. over 3 hours prior to filtration with suction, thus, white crystals is obtained. Resultant white crystal is combined and washed extensively with distilled water and further suction 3 times before it is dried under vacuum condition (setting temperate<80° C.). In this way, another 0.44 g white crystal of compound (1) is obtained from filtrate with yield rate of 91.5%.

Thus white crystal compound (1) prepared by the above-described method in total is 9.80 g homogeneous compound with yield of 91.5%.

### EXAMPLE 2

Clarithromycin 7.4 g (0.01 mole) is added into flask containing 72 g (1.0 mole) of acetone, mixing, then 1.30 g (0.01 mole) of anhydrous propionic anhydride is added thereinto with stirring at rate of 120 rpm. The reaction temperature was about 35° C. Two hours later solution becomes clear and the reaction was stopped.

10% (by weight) aqueous solution (65 ml) of sodium dodecyl sulfate supplemented with 1 g of glacial acetic acid (0.016 mole) is added dropwise at room temperature. The dropping was carried out intermittently with 2 to 5 minutes interval every 2-4 ml, and stirring quickly. The reaction remains 2.5 hours after dropping completed and large amount of white crystal appears. Recovering white crystal by filtration with suction.

The filtrate is further concentrated under reduced pressure to extract organic solvent butanone. After that, 130 ml (2 volume) of distilled water is added into concentrated solution, then mixed, and referigarited over 12 hours prior to filtration with suction, thus, white crystals is obtained. The white crystals is combined, and washed with distilled water and further suction 5 times before it is dried in vacuum desiccator (setting temperate <80 ° C.).

Finally, 3.72 g white crystal of compound (I) is obtained with yield 34.8%.

### EXAMPLE 3

### Characterization of Compound (I)

1) Physical & Chemical Property

As described hereinabove, the compound (I) of present invention is white, needle-shape crystal, which is inodorous, freely soluble in acetone, ethanol, methanol and chloroform while almost insoluble in water.

The melting point of compound (I) of present invention is 141-145° C. as digital melting point analyzer (Model WRS-1, Shanghai physical optics equipment factory) determined with temperature increased 2.5-3.0° C./min according to the method described in ANNEX VIC (on the measurement of melting point) to Chinese Pharmacopoeia (2000 edition).

The pH value of compound (I) of present invention is 5-7 as pH value analyzer (Model PHS-3C, Shanghai leici equipment factory) determined according to the method described in ANNEX VIH (on the measurement of pH value) to Chinese Pharmacopoeia (2000 edition), wherein compound (I) of present invention 0.4 g is added into 10 ml water and stirring extensively.

2) Analysis Result of High Performance Liquid Chromatography (HPLC)

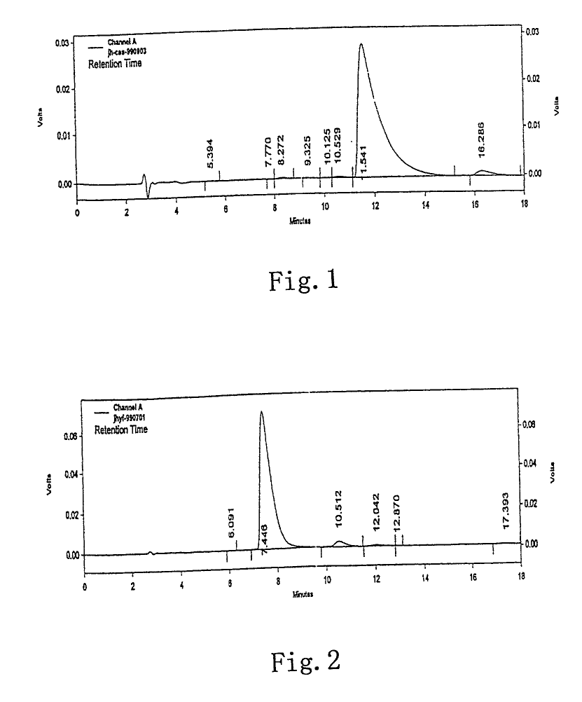

The compound (I) was analyzed by high performance liquid chromatography (HPLC) using LC-10 Model ATVP (Shimadzu Corp., Japan) HPLC analyzer, in which octadecyl silane-bound silica is used as filling material, KH₂PO₄ (0.067 mol/L): HCOH (pH is adjusted to 4.0 by H₃PO₄) as mobile phase with ratio of 35:65. Monitor wavelength is set at 210 nm with column temperature at 50° C.The retention time of compound (I) is 11.6 minute (see FIG. 1), the calculated peak area is 96.2%. In contrast, the retention time of clarithromycin is 7.5 minute, and the corresponding peak area is 0 (see FIG. 2).

This result indicates that the reaction by “one-pot” method of this invention is well completed, and the purity of said compound is greater than 96%. The titer of compound (I) of present invention is equivalent to 672.8 clarithromycin units according to the definition in the Chinese Pharmacopoeia (2000 edition), wherein 1000 clarithromycin units corresponds to 1 mg C₃₈H₆₉NO₁₃ (clarithromycin) as recorded.

3) Analysis Result of Infrared Spectroscopy (IR)

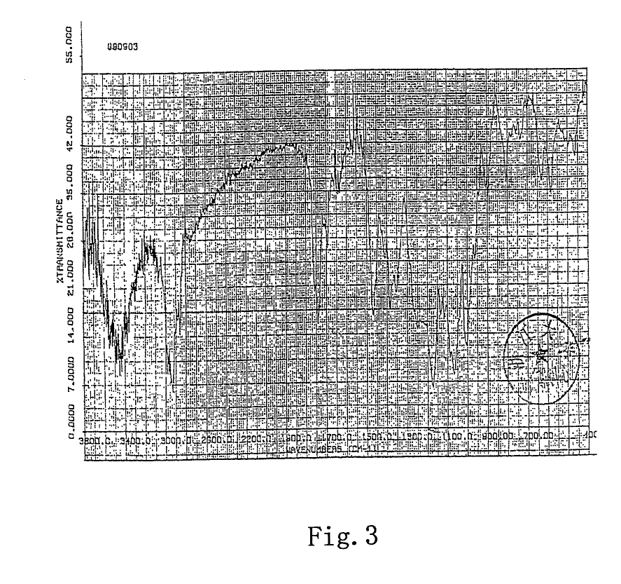

The main characteristic absorption bands of compound (I) of present invention as measured (NicoLet 5 DX, KBr tablet, resolution 4 cm⁻¹, spectrum range from 400-4000 cm⁻¹) are 3475, 2930, 2833, 1736, 1695, 1380, 1250, 1174, 1066, 1005 cm-1 (see FIG. 3).

The illumination to IR is listed as follows:

| | | | |
|---|---|---|---|
| 3475 cm⁻¹ | γ-H | hydroxy | -OH |
| 2930 cm⁻¹ | γ-H | methyl | -CH3 |
| 2833 cm⁻¹ | γ-H | methoxy | -OCH3 |
| 1738 cm⁻¹ | γ-O | lactonic ketone | C═O |
| 1695 cm⁻¹ | γ═O | cyclic ketone (saturated ketone) | |
| 1380 cm⁻¹ | δ-11 | methyl | -CH3 |
| 1250 cm⁻¹ | γ-O | propionyloxy | -OCOCH2CH3 |
| 1174 cm⁻¹ | δ-H | tertiary alcohol | |
| 1066 cm⁻¹ | γ-O | dodecyl sulfate | |

4) Analysis of nuclear magnetic resonance (NMR)

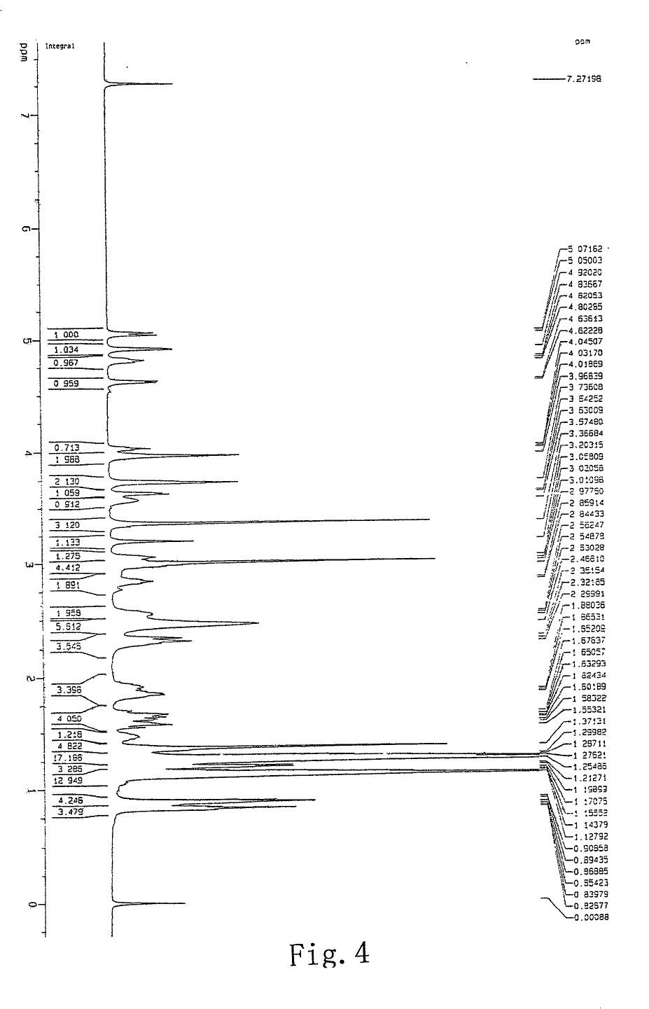

Thus, analysis of nuclear magnetic resonance (NMR) to the compound (I) of present invention is further carried out using standard method (AVANCE DMX 500 NMR Analyzer, Swissland )(see FIG. 4). The results are listed in Table 1 as follows:

TABLE 1
¹H-NMR (CDCl₃) data

| Serial | | | | | |
|---|---|---|---|---|---|
| No. | | Pro- | | | Pro- |
| of | Chemical | ton | | Chemical | ton |
| Pro- | Shifts | Num- | Serial No. | shifts | Num- |
| ton | (ppm) | ber | of Proton | (ppm) | ber |
| 2 | 2.85 | 1 | 2′ | 3.20 | 1 |
| 3 | 3.74 | 1 | 3′ | 2.47 | 1 |
| 4 | 1.81 | 1 | 5′ | 3.57 | 1 |
| 5 | 3.63 | 1 | 6′ | 1.20 | 3 |
| 7 | 1.66, 1.87 | 2 | 3N(CH₃)₂ | 2.54 | 6 |
| 8 | 2.55 | 1 | 6-OCH₃ | 3.04 | 3 |
| 10 | 2.99 | 1 | -OC(═O)CH₂CH₃ | 2.32, 1.37 | 2, 3 |
| 11 | 3.74 | 1 | 1″ | 4.92 | 1 |
| 13 | 5.06 | 1 | 2″ | 1.61, 2.32 | 2 |
| 15 | 0.85 | 3 | 3″-OCH₃ | 3.37 | 3 |
| 16 | 1.18 | 3 | 3″-CH₃ | 1.25 | 3 |
| 18 | 1.30 | 3 | 4″ | 3.04 | 1 |
| 19 | 1.16 | 3 | 5″ | 4.03 | 1 |
| 20 | 1.13 | 3 | 5″-CH₃ | 1.30 | 3 |
| 21 | 1.12 | 3 | CH₃(CH₂)₁₀CH₂—OSO₃H | 0.83, 3.96 | 3, 2 |
| 1′ | 4.58 | 1 | | | |

### EXAMPLE 4

### Antibacterical Activity In Vitro Comparison Assay and Acute Toxicity Animal Trial of Compound (I)

The primary antibacterial activity in vitro comparison assay between compound (I) (named CES hereafter) and clarithromycin are performed by No. 1 Hospital of Zhejiang Medical University. The agar dilution method, according to the method recognized by NCCLS (“National clinical analysis criterion protocol” (2^{nd} edition), Ye Yingwu and Wang Yusan ed., p562, Southeast University Press), is taken in the following pharmaceutical sensitivity tests against Streptococcus herbarum, Streptococcus haemolyticus, Staphylococcus aureus, Enterococcus faecalis, Staphylococcus epidermidis, Listeria monocytogenes, and ATCC 25923. The results is showed in table 2 as follows (unit: μg/ ml):

TABLE 2

| Antibiotics | Range of MIC | MIC₅₀ | MIC₉₀ |
|---|---|---|---|
| Clarithromycin, | 0.0125 to ≧ 128 | 0.25 | ≧128 |
| Batch No. 990701* | | | |
| CES, Batch No. 990903 | 0.0125 to ≧ 128 | 0.25 | ≧128 |
| CES, Batch No. 990901 | 0.0125 to ≧ 128 | 0.25 | ≧128 |

*Both CES 990903# and CES 990901# are derived from clarithromycin 990701#.

The results indicated that the antibacterial activity of compound (I) is comparable to that of clarithromycin without reducing pharmaceutical efficacy.

Acute toxicity animal trial to compound (I) of the present invention is carried out by using 20 KM mice (10 males) weighted 18-22 g. It is observed in acute toxicity animal trial that acute toxicity of compound (I) orally administrated to mice was quite low with LD₅₀>7.5 g/kg. Subsequently, the maximal tolerance dose to mice is greater than 8 g/kg as maximal tolerance test determined. After being administrated with compound (I), the mice become furriery flab, drowsiness, spiritless and reduced movement, but no death was observed. The mice restored gradually 3 days after administration of compound (I).

In contrast, the LD₅₀ of clarithromycin orally administrated to mice and rat is 2.7 to 3.5 g/kg, and the LD50 of etothromycin orally administrated to mice is above 6.45 g/kg according to Current Structural Pharmaceutics Collection, Wang Zeming ed., Beijing Science and Technology Press, p208-210.

These results further indicate that compound (I) has little toxicity and therefore is safe to human.

While the invention has been described with respect to the above specific embodiments, it should be recognized that various modifications and changes may be made to the invention by those skilled in the art which also fall within the scope of the invention as defined by the appended claims.

What is claimed is:

1. A macrolide derivatives, an acid salt of 2′-propionate clarithromycin, wherein the compound has molecular formula C₄₁H₇₃NO₁₄·R with general formula as follows:

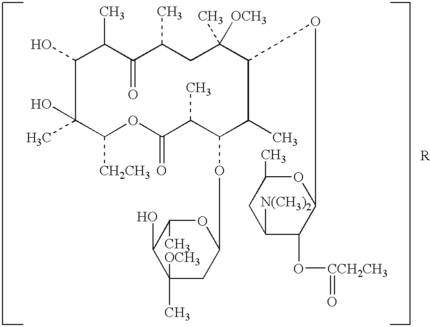

2. A compound of claim 1, wherein R is selected from the group consisting of dodecyl sulfate, thiocyanic acid, glucoheptose, ascorbic acid, and fumaric acid.

3. A compound of claim 1, wherein R is dodecyl sulfate with structural formula I as follows

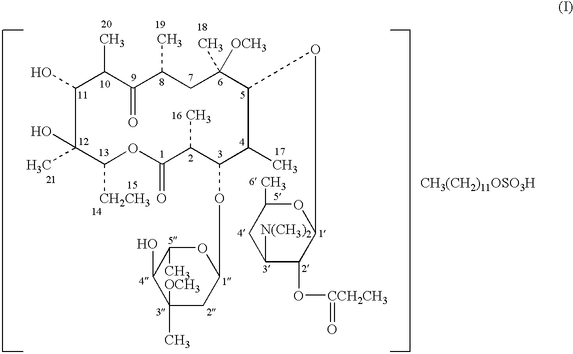

4. A method for preparing the compound (I) of claim 3 which is characterized by propionylizing the 2′-hydroxyl group of clarithromycin to produce 2′-propionyl clarithromycin (II), followed by reacting it with sodium dodecyl sulfate to form compound (I).

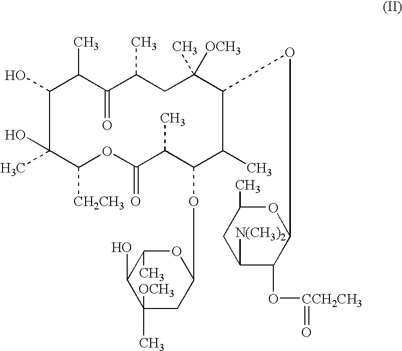

5. The method of claim 4 which is characterized by that
1) adding claithromycin into a water-soluable non-polar solvent under anhydrous condition, then adding anhydrous propionic anhydride or propionyl chloride thereinto with temperature held at 15 to 45° C., stirring for 0.5 to 3 hours to make clarithromycin fully dissolved and thereby obtaining clear solution of 2′-propionyl clainthromycin,
2) reducing the temperature of solution to about 10-35° C., keeping at the temperature and adding dropwise aqueous solution of sodium dodecyl sulfate into said solution with stirring over 30 to 90 minute, stopping reaction, then
3) filtering with suction to obtain white crystal,
4) concentrating the resulting filtrate and reclaiming solvent, adding 0.2 to 3 volumes distilled water into the concentrated solution followed by refrigerating at 0 to 10° C. over 1-24 hours before filtering said solution with suction to obtain white crystal,
5) pooling resultant white crystal in step 3) and 4), then wasing with distilled water extensively, and drying under vacuum condition to obtain compound (I).

6. The method of claim 5 for preparing compound (I) which is characterized by that both propionylizing 2′-hydroxyl group of clarithromycin and the subsequent salifying with sodium dodecyl sulfate are carried out in “one-pot” method.

7. The method of claim 5 for preparing compound (I) which is characterized by that said water-soluable non-polar solvent used in step 1) is selected from the group consisting of acetone, butanone, cyclohexanone, and tetrahydrofuran and the like, preferably is acetone.

8. The method of claim 5 for preparing compound (I) which is characterized by that said aqueous solution of sodium dodecyl sulfate optionally further includes an acid agent selected from the group consisting of acetic acid, propionic acid, and sulfuric acid and the like.

9. The method of claim 5 for preparing compound (I) which is characterized by that adding dropwise is carried out continuously or intermittently.

10. The method of claim 5 for preparing compound (I) which is characterized by that the amount of each reaction agent used is based on 1 mole clarithromycin, anhydrous propionic anhydride or propionyl chloride added is from 0.5 to 2 moles, sodium dodecyl sulfate is from 1 to 4 moles.

11. A pharmaceutical composition comprising 2′-propionate clarithromycin dodecyl sulfate used as antibacterials and/or antipyrotics.
